(19) United States
(12) Patent Application Publication
(10) Pub. No.: US 2024/0116849 A1
(43) Pub. Date: Apr. 11, 2024

(54) METHOD OF MANUFACTURING HALOGEN OXOACID AND MANUFACTURING APPARATUS THEREFOR

(71) Applicant: TOKUYAMA CORPORATION, Yamaguchi (JP)

(72) Inventors: Akihiro SAITO, Yamaguchi (JP); Naoto MOCHIZUKI, Yamaguchi (JP)

(73) Assignee: TOKUYAMA CORPORATION, Yamaguchi (JP)

(21) Appl. No.: 17/920,921

(22) PCT Filed: Jun. 7, 2022
(86) PCT No.: PCT/JP2022/022882

§ 371 Date: Oct. 24, 2022

(30) Foreign Application Priority Data
Jun. 7, 2021 (JP) 2021-094936

(51) Int. Cl. C07C 209/68; B01J 19/02; B01J 19/24

(52) U.S. Cl. CPC C07C 209/68; B01J 19/02; B01J 19/243; B01J 2219/00033; B01J 2219/00166; B01J 2219/0295; B01J 2219/182

(57) ABSTRACT

Provided are a manufacturing method and a manufacturing apparatus for efficiently manufacturing a halogen oxoacid solution with a high quality and excellent industrial properties. Specifically, a manufacturing method and a manufacturing apparatus of a halogen oxoacid are provided. The manufacturing method and the manufacturing apparatus include continuously supplying an organic alkaline solution and a halogen from a first end to a second end of a reaction tube so that liquid phase parts and/or gas phase parts are alternately and repeatedly provided in a transfer passage of the reaction tube, to perform gas-liquid mixing of the organic alkaline solution and the halogen at the liquid phase parts and/or gas phase parts.

### TECHNICAL FIELD

The present invention relates to a method of manufacturing a halogen oxoacid solution having excellent industrial properties, in which an organic alkaline solution and a halogen are simultaneously supplied to a reaction tube and dissolved by gas-liquid mixing in the tube.

### BACKGROUND ART

Various known schemes have been attempted as methods of mixing a target liquid and a gas that are soluble in the target liquid to realize an intended treatment. Examples include a method (Patent Document 1) in which gas is absorbed while circulating a liquid in a tank to obtain a liquid having a predetermined concentration. There is also known an apparatus having a tubular shape instead of using a tank, with the aim of increasing efficiency. Examples include ozone injection into flowing water to produce ozone water (Patent Document 2) and carbon dioxide injection into alkali waste water for a neutralization treatment (Patent Document 3).

### PRIOR ART DOCUMENTS

### Patent Document

Patent Document 1: JP 2005-21798 A
Patent Document 2: JP 2010-221180 A
Patent Document 3: JP 53-118278 A

### SUMMARY OF INVENTION

### Technical Problem

The gas dissolution methods described above can work well as long as the amount of gas used can be completely dissolved in the liquid, but these methods do not work satisfactorily as a process for dissolving, in a liquid, a gas supplied at a high flow rate. That is, when a gas is used that has low solubility in the target liquid, the undissolved gas leaks out of the system, and thus, the gas is consumed more than necessary. If a gas that is harmful to the environment, such as ozone and a halogen, leaks out of the system, countermeasures against leakage need to be taken in order to detoxify the gas. Examples of methods for increasing the solubility of the gas in the liquid include a method of increasing the time and frequency of contacts between the gas and the liquid. However, for this purpose, methods such as increasing the volume of the apparatus, and providing a line mixer intended for stirring during the process, need to be employed. In all of these methods, the size of the apparatus increases and the structure becomes complicated, and thus, the cost and quality are also strongly affected.

Therefore, the present invention focuses on the solubility of a halogen in an organic alkaline solution, and in the present invention, the halogen and the organic alkaline solution are subjected to gas-liquid mixing in the tube to dissolve the halogen in the organic alkaline solution, without using a line mixer. According to the present invention, there is no need to increase the size of the apparatus and to use a complicated structure. Furthermore, according to the present invention, it is possible to provide a manufacturing method and a manufacturing apparatus for efficiently manufacturing a halogen oxoacid solution with a high quality and excellent industrial properties, which can reduce unreacted halogen.

### Solution to Problem

As a result of diligent studies to achieve the object described above, the present inventors have found a method and a manufacturing apparatus for industrially manufacturing a halogen oxoacid more stably and efficiently, in which gas-liquid mixing of an organic alkaline solution and a halogen is performed at liquid phase parts and/or gas phase parts by continuously supplying the organic alkaline solution and the halogen from one end to the other end of a reaction tube, and alternately providing the liquid phase parts and the gas phase parts repeatedly in a transfer passage of the reaction tube, and which led to the completion of the present invention.

That is, the present invention is configured as follows.
Aspect 1: A method of manufacturing a halogen oxoacid, the method including continuously supply an organic alkaline solution and a halogen from the first end to the second end of the reaction tube so that liquid phase parts and gas phase parts are alternately and repeatedly provided in a transfer passage of the reaction tube, to perform gas-liquid mixing of the organic alkaline solution and the halogen at the liquid phase parts and/or gas phase parts.
Aspect 2: The method of manufacturing a halogen oxoacid according to Aspect 1, in which a ratio of a volume flow rate of the halogen to a volume flow rate of the organic alkaline solution supplied to the reaction tube is from 1 to 50.
Aspect 3: The method of manufacturing a halogen oxoacid according to Aspect 1 or 2, in which the reaction tube has an axis extending from the first end to the second end of the reaction tube and extends in an axial direction while circling around the axis.
Aspect 4: The method of manufacturing a halogen oxoacid according to any one of Aspects 1 to 3, in which the reaction tube is arranged to extend substantially in a horizontal direction.
Aspect 5: The method of manufacturing a halogen oxoacid according to any one of Aspects 1 to 4, in which the reaction tube has an axis extending from the first end to the second end of the reaction tube, and the reaction tube is formed in a spiral shape having a spiral axis coinciding with the axis.
Aspect 6: The method of manufacturing a halogen oxoacid according to any one of Aspects 1 to 5, in which the reaction tube contains a fluororesin.
Aspect 7: The method of manufacturing a halogen oxoacid according to any one of Aspects 1 to 6, in which an average inner diameter of the reaction tube is 5 mm or greater.
Aspect 8: A halogen oxoacid manufacturing apparatus, including a reaction tube having an axis extending from a first end to a second end of the reaction tube, the reaction tube extending in an axial direction while circling around the axis, in which the reaction tube with the axis extending substantially in a horizontal direction is configured to continuously supply an organic alkaline solution and a halogen from the first end to the second end of the reaction tube so that liquid phase parts and gas phase parts are alternately and repeatedly provided in a transfer passage of the reaction tube, to perform gas-liquid mixing of the organic alkaline solution and the halogen at the liquid phase parts and/or gas phase parts.
Aspect 9: The halogen oxoacid manufacturing apparatus according to Aspect 8, further including a supplying unit configured to supply, to the reaction tube, the organic alkaline solution and the halogen with a ratio of a volume flow rate of the halogen to a volume flow rate of the organic alkaline solution from 1 to 50.
Aspect 10: The halogen oxoacid manufacturing apparatus according to Aspect 8 or 9, in which the reaction tube is formed in a spiral shape, and a spiral axis of the reaction tube extends substantially in the horizontal direction.
Aspect 11: The halogen oxoacid manufacturing apparatus according to any one of Aspects 8 to 10, in which the reaction tube contains a fluororesin.
Aspect 12: The halogen oxoacid manufacturing apparatus according to any one of Aspects 8 to 11, in which an average inner diameter of the reaction tube is 5 mm or more.
Aspect 13: The halogen oxoacid manufacturing apparatus according to any one of Aspects 8 to 12, in which the reaction tube is configured to achieve a liquid retention time of the organic alkaline solution of 5 seconds to 30 minutes.

### Effects of Invention

An organic alkaline solution and a halogen are continuously supplied from a first end of a reaction tube, and a reaction solution containing the generated halogen oxoacid is continuously removed from a second end of the reaction tube. In the mixed liquid in the reaction tube, gas-liquid mixing frequently occurs, and thus, the component concentrations of the organic alkaline solution, which is the raw material, and the generated halogen oxoacid, the pH, and the like are maintained constant in a steady state. Therefore, side reactions and the like are suppressed, and a reaction solution having good storage stability is obtained. Further, the amount of unreacted halogen is reduced, and thus it is possible to suppress waste of the raw materials. In addition, an effect of reducing the time from the start of the reaction until a reaction solution having a stable composition is obtained is also achieved, and thus, the generated amount of waste solution can also be reduced. Therefore, it is possible to efficiently and stably obtain a halogen oxoacid. In addition, by the continuous supply of the raw materials and the continuous removal of the reaction products, it is possible to realize industrial mass production.

### BRIEF DESCRIPTION OF DRAWINGS

FIG. 1 is a schematic view illustrating one aspect of a manufacturing apparatus according to an embodiment of the present invention.

FIG. 2 is a graph showing a relationship between operation time and effective chlorine concentration when a manufacturing method according to an embodiment of the present invention and a comparative example is used.

FIG. 3 is a schematic view illustrating an existence state of gas phase parts and liquid phase parts in a reaction tube. (a) illustrates a state where the liquid phase parts and the gas phase parts are alternately and repeatedly provided in the reaction tube, and (b) illustrates a state where the liquid phase parts and the gas phase parts are not alternately and repeatedly provided in the reaction tube.

### DESCRIPTION OF EMBODIMENTS

### Method of Manufacturing Halogen Oxoacid

### Reaction Scheme and Reaction Tube

One feature of the present embodiment is use of a scheme in which an organic alkaline solution and a halogen are continuously supplied, and a reaction solution containing produced halogen oxoacid is continuously removed. In the continuous removal of the reaction solution including the halogen oxoacid, it is preferable to remove an amount corresponding to the amount of the organic alkaline solution and the halogen continuously supplied. The corresponding amount refers to an amount equal to or proportional to the total amount of the supplied organic alkaline solution and the halogen (total amount of organic alkali and halogen>removal amount; both in volume). The same applies to the following description of the manufacturing apparatus.

In the present invention, it is preferable to maintain constant the amount of the organic alkaline solution and the produced halogen oxoacid, which are components in the reaction tube in a steady state, and the pH and the like of the reaction solution removed from the reaction tube, and in a preferable aspect, the supply amount is adjusted with high accuracy.

In a known semi-batch type reaction scheme, a process of adding a halogen to an organic alkaline solution charged in a reactor is used, and thus, the pH of the reaction solution tends to be high at an early stage of the reaction, and at a high pH, decomposition of the halogen oxoacid easily occurs. In addition, in the halogen oxoacid produced in the high pH region, the decomposition product is produced as a result of decomposition of the halogen oxoacid, which causes a problem in storage stability. In contrast, if the organic alkaline solution and the halogen are continuously supplied and the reaction solution is continuously removed, the reaction solution in the reaction tube reaches a steady state after a certain period of time, and thereafter, and the pH is maintained at a constant level. In this stable state, decomposition of the halogen oxoacid is suppressed.

The manufacturing method according to the embodiment of the present invention can prevent persistently high pH of the reaction solution. The pH in the present invention is a value at 25° C. unless otherwise indicated.

Employing the above-described scheme in which the organic alkaline solution and the halogen are continuously supplied and the reaction solution containing the produced halogen oxoacid is continuously removed makes it possible to reduce the retention time under the high pH condition at which the strongest side reaction in the reaction occurs. Examples of the halogen to be used include chlorine gas and chlorine, and when chlorine gas or chlorine is used, the chlorine yield can be maintained at a high level. The chlorine yield mentioned here can be determined from the ratio (%) of the number of moles of hypochlorite ions produced relative to the number of moles of chlorine molecules supplied into the organic alkaline solution. When the whole amount of chlorine added to the organic alkaline solution undergoes reaction (and no decomposition occurs), the chlorine yield is 100%. The chlorine yield decreases when hypochlorite ions decompose during the reaction.

In the present invention, the reaction tube refers to an apparatus in which a chemical reaction occurs in a process of manufacturing a chemical substance. Preferably, the organic alkaline solution and the halogen are continuously supplied to the reaction tube at a constant rate. A supply at a constant rate means that a supply speed is constant. The continuous removal of the reaction solution may not start simultaneously with the start of the manufacturing method according to the embodiment of the present invention, and may be performed when the pH of the reaction solution removed from the reaction tube is constant.

In addition, in a preferable aspect, the components in the reaction tube and the pH of the reaction solution are uniform. In the reaction tube, the pH of the reaction solution of the organic alkaline solution and the halogen being supplied is preferably from 10.5 to 14.5. Furthermore, the pH of the reaction solution of the organic alkaline solution and the halogen is more preferably from 10.5 to 13.8. The pH of the reaction solution of the organic alkaline solution and the halogen is even more preferably from 12.0 to 13.8. The pH of the reaction solution removed from the reaction tube is preferably from 12.0 to 13.8.

In order to mix the organic alkaline solution and the halogen in the reaction tube, it is preferable that liquid phase parts and gas phase parts are alternately and repeatedly provided in a transfer direction in a transfer passage. Thus, the liquid can be stirred and mixed uniformly without installing a line mixer for a mixing operation in the tube. The line mixer (a stirring and mixing device) is normally employed as a stirring means to achieve uniformity of the fluid passing through the tube. Examples of the line mixer mainly include a mixer that drives a stirring blade installed in a space in a tube to mix a fluid, or an in-line mixer (stationary type mixing and stirring device) that does not include a drive unit and mixes the fluid by passing the fluid through elements fixed in the space in the tube. These line mixers are used for efficient mixing of fluids. However, in case of production of a high-purity liquid, such as a semiconductor chemical liquid, in which contamination by particles, metal components, etc., should be avoided, it may be difficult to provide a line mixer in a tube, because contamination from a contact point with the liquid significantly affects quality. In a reaction tube without a line mixer, the degree of gas-liquid mixing will be lowered, which results in non-uniform concentration of the reaction solution and generation of a short path between the gas phase parts in the tube. Thus, using such a reaction tube in an industrial manufacturing method is disadvantageous.

When the liquid phase parts and the gas phase parts in the transfer passage of the reaction tube are not alternately and repeatedly provided in the transfer direction, the number and the frequency of contacts between the gas and the liquid decrease, and thus, the mixed state of the gas and the liquid in the tube deteriorates. In this case, in order to stabilize the liquid composition, it is necessary to add a process of improving the mixed state of the gas and the liquid, and if no line mixer is installed in the tube, the length of the reaction tube needs to be extended. When the length of the reaction tube is increased, equipment costs increase and an amount of waste solution generated during replacement of the liquid in the reaction tube increases, and thus, there is a disadvantageous effect on costs both regarding equipment and raw materials. When the liquid phase parts and the gas phase parts are alternately and repeatedly provided in the transfer passage of the reaction tube, as in the present invention, the number of times and frequency of contacts between the gas and the liquid are finely distributed in the reaction tube. This makes it possible to create a state close to a pseudo plug flow in the reaction tube, and thus, to continuously obtain a reaction solution having uniform concentration. As a result, it is possible to uniformly mix the organic alkaline solution and the halogen in the reaction tube and to eliminate the need for installing the line mixer.

As a result of these effects, the present invention is also characterized in that a liquid with a stable composition is continuously obtained only by replacing the reaction solution in the reaction tube once. That is, the volume required for replacing the unreacted organic alkaline solution with the reacted liquid in the tube depends on the length from an inlet to an outlet of the reaction tube. Thus, it is possible to optimize a replacement operation of the liquid in the tube with the reaction solution, and the amount of waste solution to be discarded which is generated from the start of the reaction until the liquid composition is stable can be minimized. This is industrially advantageous effect.

The simplest method for providing the liquid phase parts and the gas phase parts alternately in the reaction tube is to choose a tube diameter of a supply tube for supplying the gas phase and the liquid phase to have a diameter equal to or smaller than the diameter of the reaction tube. This makes it possible to alternately create gas phase parts and liquid phase parts having the same diameter as the reaction tube on an inlet side of the reaction tube in which the gas and the liquid contact each other. However, when the liquid-gas ratio (a value obtained by dividing the volume flow rate of the gas phase part supplied to the reaction tube per time unit by the volume flow rate of the liquid phase part supplied to the reaction tube per time unit) is 1 or greater, the halogen present in the gas phase part is not sufficiently dissolved in the liquid phase part, and is emitted as undissolved halogen from an outlet side of the reaction tube. An example of a countermeasure includes a method of extending the length of the reaction tube to increase the time for the halogen to dissolve. However, in a simpler method of promoting the dissolution of the halogen in the reaction tube, the tube preferably extends to circle with respect to an axial direction in which the reaction tube extends. That is, when a direction extending from a first end to a second end of the reaction tube is defined as an axis, a configuration is preferable in which the reaction tube extends in the axial direction while circling around the axis. The axial direction described here preferably extends in a horizontal direction rather than a vertical direction, but the axis is not limited to the horizontal direction. The direction in which the axis extends can be inclined, and even if the axial direction is inclined, it is more preferable that the reaction tube extends in a substantially horizontal direction.

The diameter of the revolution required for circling in the axial direction can be determined depending on the length of the reaction tube and the strength of the material used for the reaction tube, and examples thereof include diameters of 30 mm or more and 3000 mm or less, and preferably 60 mm or more and 600 mm or less. When the reaction tube circles one or more times, an effect of improving the mixing efficiency between the gas and the liquid is obtained. The inside of the reaction tube is basically divided into two phases including the gas phase part that rises by the buoyancy generated according to the volume of the gas, and the liquid phase part that descends by the gravity. However, when the reaction tube circles, for example, the gas phase part can contact at least once the liquid phase part accumulated in a lower part of the reaction tube in the vertical direction. Therefore, if the number of revolutions of the reaction tube is high, it is advantageous for the mixing between the gas and the liquid, and generation of a short path between the gas phase parts can be prevented. It is noted that the gas phase part may not be present in the lower part of the reaction tube in the vertical direction. Furthermore, the lengths in the transfer direction of the gas phase parts and the liquid phase parts present in the reaction tube may not be uniform. The number of revolutions of the reaction tube has no upper limit, but is preferably 2 or more revolutions, more preferably 5 or more revolutions, and even more preferably 10 or more revolutions. On the other hand, the upper limit of the number of revolutions is usually 50 revolutions or less. In addition, the range of the average inner diameter of the reaction tube has no upper limit, but is preferably 5 mm or more, more preferably 5 mm or more and 500 mm or less, and even more preferably 10 mm or more and 100 mm or less.

In appearance, an apparatus satisfying these apparatus configurations is most preferably a reaction tube formed in a spiral shape along the axial direction. However, the configuration of the reaction tube is not limited thereto, and in other preferable configurations, the reaction tube is bent in an alternating manner along the axial direction, or the reaction tube is processed into a wavy line shape along the axial direction.

The inner diameter and the length of the reaction tube affect the volume of the reaction solution, and thus, are important to achieve the industrial mass production of the reaction solution. The industrial mass production described here means efficiently and continuously producing the target reaction solution while reducing the amount of the generated waste solution. In the industrial mass production, it is preferable to produce, per one hour, an amount of the reaction solution that is the same as or greater than the volume of the liquid filling the tube volume, more preferably an amount of 5 times or more, and even more preferably an amount of 100 times or more the volume of the liquid filling the tube volume.

An inert gas can be supplied to the reaction tube, in addition to the organic alkaline solution and the halogen that are continuously supplied to the reaction tube. The supply of the inert gas is useful both in alternately and repeatedly providing the liquid phase parts and the gas phase parts in the reaction tube, and in preventing backflow of the liquid phase parts and the gas phase parts in the system. The inert gas refers to stable gas that does not interfere in the reaction, such as air, nitrogen, argon, and helium, and air is preferred with regards to costs. However, carbon dioxide contained in the air may dissolve in the reaction solution and cause the pH to decrease, or may react with the reaction solution and generate impurities, and thus, it is more preferable to use an inert gas that is purified to a high purity level, and among purified inert gases, it is preferable to use nitrogen from the financial aspects.

The volume in the reaction tube can vary depending on the supply speed of the organic alkaline solution to be used. When a value obtained by dividing the volume of the reaction tube by the volume of the organic alkaline solution supplied to the reaction tube per time unit is defined as the liquid retention time, it is preferable that the reaction tube has such a volume that the liquid retention time of the organic alkaline solution in the reaction tube is preferably from 5 seconds to 30 minutes, and more preferably from 10 seconds to 5 minutes. In the present invention, a liquid having a stable composition can be continuously obtained only by replacing the reaction solution in the reaction tube once. This means that the time required to achieve a stable liquid composition is directly related to the liquid retention time. When the components in the tube are insufficiently mixed at the start of the reaction, the time required to obtain a liquid having a stable composition is at least three times or more the liquid retention time.

The supply speed of the halogen, expressed as a ratio of the volume flow rate of the halogen to the volume flow rate of the organic alkaline solution supplied to the reaction tube, is preferably from 1 to 50, and more preferably from 10 to 30. The supply speed of the halogen within such a range makes it possible to contribute to realization of alternate provision of the gas phase parts and the liquid phase parts in the transfer direction in the reaction tube, even when the shape of the reaction tube is not as described above, that is, even when the tube does not extend so as to circle with respect to the axial direction in which the reaction tube extends.

The volume flow rate of the halogen supplied to the reaction tube is calculated as a converted value of the halogen in gaseous form at 0° C. and 1 atm.

The dissolved gas in the solution can be degassed by causing the halogen supplied to the reaction tube to dissolve in the organic alkaline solution in the tube. Specifically, the halogen in the reaction tube produces dissolution heat that increases the temperature of the solution, dissolved gas that no longer dissolve in the liquid travels out of the liquid, and thus a degassing effect is achieved. As the liquid-gas ratio increases, the dissolution heat also increases proportionally to the supply amount of the halogen, and thus, the degassing effect is more easily obtained. Examples of the dissolved gas include nitrogen, oxygen, and carbon dioxide, but the dissolved gas to be degassed is not limited thereto.

There is a known reaction tube having a cylindrical shape (a cylindrical reaction tube), which is a reaction tube in which only one of openings is closed. The reaction tube having the cylindrical shape has a structure that is not suitable for obtaining, in a short period of time, a liquid having stable composition, which is an effect of the present invention. Specifically, in the cylindrical reaction tube, it is necessary to separately provide a location required for passing the liquid in the reaction tube, and the liquid in the reaction tube is not easily replaced, and thus it is difficult to obtain a stable liquid composition. Therefore, the liquid needs to be discarded until a stable composition is achieved. However, in the present invention, a stable composition is achieved by replacing the liquid once. Thus, the amount of generated waste liquid can be reduced and the present invention is economically advantageous.

### Organic Alkaline Solution

The organic alkaline solution supplied to the reaction tube can be an aqueous solution in which an organic alkali is dissolved in water or a solution in which an organic alkali is dissolved in a non-aqueous solvent. The organic alkaline solution can be obtained by dissolving an organic alkali in water or a non-aqueous solvent or diluting a commercially available organic alkaline solution to a desired concentration. Among water and the non-aqueous solvent, water is preferably used from the viewpoint that water is easily obtained in industrial fields and organic alkaline solutions of high purity can be produced. Examples of the non-aqueous solvent include known organic solvents in which organic alkalis can be dissolved. Specific examples of the non-aqueous solvent include alcohols and glycols, and particularly preferred are methanol and propylene glycol. The concentration of the organic alkaline solution is not particularly limited, but when the concentration of the organic alkali is high, salts precipitate and the solution becomes solid. Thus, the concentration of the organic alkali in the organic alkaline solution is preferably from 0.01 to 30 mass %, more preferably from 0.05 to 27.5 mass %, and even more preferably from 0.1 to 25 mass %.

The solvent used in the organic alkaline solution can be an aqueous solution in which only water is used as the solvent, can be a non-aqueous solution obtained by mixing with an organic solvent, and can be a solvent obtained by mixing an aqueous solution with an organic solvent. The solvent can be appropriately changed depending on the application of the solution containing the halogen oxoacid. For example, when ruthenium is to be cleaned, an organic alkaline aqueous solution can be used, because using water only as the solvent can result in sufficient cleaning.

In the present embodiment, the organic alkaline solution is preferably a solution of an onium hydroxide, and examples of the onium hydroxide include one or more selected from the group consisting of ammonium hydroxide, phosphonium hydroxide, sulfonium hydroxide, iminium hydroxide including multiple bonds, and diazonium hydroxide. Among these, the organic alkaline solution is more preferably a solution of ammonium hydroxide in which a large amount of relatively stable compounds are present. The solution of the onium hydroxide described above is preferably an aqueous solution of an onium hydroxide. Furthermore, the solution of the ammonium hydroxide described above is preferably a quaternary alkylammonium hydroxide solution.

The quaternary alkylammonium hydroxide solution is preferably a solution of quaternary alkylammonium hydroxide in which each alkyl group independently has carbon number from 1 to 10, and more preferably, a solution of quaternary alkylammonium hydroxide in which each alkyl group independently has carbon number from 1 to 5. Specific examples of the quaternary alkylammonium hydroxide include tetramethylammonium hydroxide, tetraethylammonium hydroxide, tetrabutylammonium hydroxide, and choline. One type of these quaternary alkylammonium hydroxides can be used alone, or two or more types can be used in combination. The four alkyl groups contained in the quaternary alkylammonium hydroxide can have the same number of carbons, or can have a different number of carbons.

The various conditions in the description above and below, such as the concentration range of the organic alkali in the organic alkaline solution supplied to the reactor, the range of the pH of the organic alkaline solution, the concentration range of the organic alkali in the reaction solution, and the like can also be applied when using any of the specific examples of the organic alkalis described above.

The organic alkaline solution can include chemical species other than the organic alkali. For example, the chemical species is an organic material containing a halide and a halogen. Specific examples of the organic material include tetraalkylammonium halides such as tetramethylammonium bromide, but the organic material is not limited thereto. Furthermore, the reaction solution obtained as a result of the reaction with a halogen can be recycled as the organic alkaline solution, and can be supplied with the same halogen or a different halogen.

### Process of Manufacturing Reaction Solution Containing Halogen Oxoacid by Gas-Liquid Mixing of Organic Alkaline Solution and Halogen

In a process of mixing an organic alkaline solution and a halogen by gas-liquid mixing and reacting the organic alkaline solution and the halogen to manufacture a reaction solution containing a halogen oxoacid, the pH of the reaction solution containing the halogen oxoacid generated in the reaction tube tends to decrease. In the present embodiment, a lower limit of the pH of the organic alkaline solution which is the raw material is 10.5 or more, preferably 11.0 or more, even more preferably 11.5 or more, and particularly preferably more than 12.0. An upper limit of the pH of the organic alkaline solution depends on the concentration of the organic alkali. Examples of the upper limit of the pH of the organic alkaline solution include a pH of 14.5 or less.

The organic alkaline solution used in the present embodiment is preferably a solution in which the content of metals, specifically, sodium, potassium, aluminum, magnesium, iron, nickel, copper, silver, cadmium, and lead is 0.01 ppb more and 20 ppb or less. Needless to say, the content of the metal contained in the organic alkaline solution used can be less than 0.01 ppb, but it is difficult to obtain such an organic alkaline solution.

Therefore, when an organic alkaline solution in which the content of the above-described metals satisfies the range mentioned above is used, the organic alkaline solution can be easily obtained, and it is easy to remove or reduce the metal impurities during and after the manufacturing of the reaction solution including the halogen oxoacid.

The above-described metals are eluted at a location where the organic alkaline solution is in contact with the reaction tube, and thus, it is preferable to provide a reaction tube having a small contact surface area. That is, using a reaction tube having a small volume is an important factor for reducing impurities, and is very effective in terms of quality control. The contact surface area of the reaction tube has no upper limit, but a suitable contact surface area is 0.01 m² or more and 10 m² or less, and more preferably, from 0.1 m² or more and 1.0 m² or less.

It is possible to use a commercially available organic alkaline solution as the organic alkaline solution described above. Among the commercially available organic alkaline solutions, an organic alkaline solution used as a photoresist developing solution for a semiconductor element that is refined to high purity by using an electrolytic method, and/or by contact with an ion exchange resin, and the like, can be suitably used. These commercially available organic alkaline solutions can be used after being diluted in a solvent that does not include metal impurities, such as ultrapure water.

In the manufacturing method according to the embodiment of the present invention, the supply speed of the organic alkaline solution used is preferably from 33 mL/min to 12 L/min, and more preferably from 0.2 L/min to 6 L/min, when the volume of the reaction tube is 1 L.

### Reaction Occurring by Contact Between Organic Alkaline Solution and Halogen

For example, when the quaternary alkylammonium hydroxide is used as the organic alkali, and a solution thereof is brought into contact with a halogen to cause a reaction, the hydroxide ions of the quaternary alkylammonium hydroxide are replaced with hypochlorite ions produced by the halogen, and a quaternary alkylammonium hypohalite solution is produced.

In the present embodiment, the halogen used is not particularly limited, and commercially available halogens can be employed. Specific examples of the halogen include chlorine, bromine, iodine, hypochlorous acid, hypobromous acid, hypoiodous acid, chlorous acid, bromous acid, iodous acid, chloric acid, bromic acid, and iodic acid. When chlorine or bromine are used, gases thereof may be used. Among these gases, chlorine gas is preferably used.

Next, in the present embodiment, a method including using a quaternary alkylammonium hydroxide solution as the organic alkaline solution, using chlorine gas as the halogen, and contacting the quaternary alkylammonium hydroxide solution and the chlorine gas is described as an example of the embodiment of the present invention. In the following description, the quaternary alkylammonium hydroxide solution can be used as the organic alkaline solution, and the chlorine gas can be used as the halogen, without description of the reason, but this is merely an example.

### pH of Liquid Phase Part During Reaction

The pH of the liquid phase part during the reaction of the present embodiment is preferably 10.5 or more. The liquid phase part in the present embodiment is a portion formed by the reaction solution generated as a result of mixing the quaternary alkylammonium hydroxide solution and the chlorine gas during the reaction. The upper limit of the pH of the liquid phase part is not particularly limited, but if the pH is too high during the reaction and the solution is stored at the same pH for a long period of time after the reaction is completed, the hypochlorite ions may decompose and the effective chlorine concentration may decrease. Therefore, the pH of the liquid phase part during the reaction is preferably 10.5 or more and 14.5 or less, more preferably 10.5 or more and 13.8 or less, and even more preferably 12 or more and 13.8 or less. When the pH is within the range described above, decomposition of the hypochlorite ions is suppressed during storage of the obtained quaternary alkylammonium hypochlorite solution, and the storage stability is improved.

### Reaction Temperature

The range of the reaction temperature in the manufacturing method of the present embodiment is preferably −35° C. or more and 45° C. or less, more preferably −15° C. or more and 40° C. or less, and even more preferably −5° C. or more and 35° C. or less. When the reaction temperature is within the range described above, the organic alkaline solution and the halogen react sufficiently, and the halogen oxoacid can be obtained with a high yield. When the reaction temperature is less than −35° C., the organic alkali starts to solidify and does not sufficiently react with the halogen. On the other hand, when the reaction temperature exceeds 45° C., the halogen oxoacid ions generated in the halogen oxoacid solution are decomposed by the heat. In particular, when the pH during the reaction is 13.8 or more and the reaction temperature is high, the halogen oxoacid strongly decomposes. The yield of the halogen oxoacid can be evaluated by the chlorine yield. As described above, according to the manufacturing method of the present embodiment, it is possible to manufacture a halogen oxoacid that has excellent storage stability, and can maintain sufficient cleaning and removal performance, even 10 days after manufacturing, for example. As can be understood from the above, the halogen oxoacid obtained by the manufacturing method of the present embodiment has excellent storage stability, and can be suitably used in the manufacturing process of semiconductor elements.

### Material of Inner Surface of Reaction Tube

In the present embodiment, the organic alkaline solution and the chlorine gas are brought into contact with each other in the reaction tube to manufacture a halogen oxoacid. At this time, first, a predetermined amount of the organic alkaline solution is introduced into the reaction tube, and then, the chlorine gas can be introduced to come into contact with the organic alkaline solution.

In the present embodiment, a surface contacted by the organic alkaline solution in the reaction tube (hereinafter, simply referred to as “inner surface of the reaction tube”) is formed of a general-purpose borosilicate glass, or an organic polymer material. According to studies by the present inventors and the like, when a reaction tube made of general-purpose borosilicate glass (hereinafter, referred to as “made of glass”) is used as the reaction tube, metal components contained in the glass, such as sodium, potassium, and aluminum, are slightly dissolved into the organic alkaline solution. This is probably because the organic alkaline solution used as the raw material is alkaline. Therefore, the inner surface of the reaction tube is more preferably formed from an organic polymer material, so that contamination by impurities (metal impurities) including the above-described metals can be further reduced.

The reaction is preferably performed in an environment shielded from light, and specifically, the reaction tube is preferably a reaction tube having a light-shielded interior. The chlorine gas present in the reaction tube may be excited by light and generate chlorine radicals. When chlorine radicals are generated, the chlorine radicals may affect and decompose the organic alkaline solution present in the reaction tube and the halogen oxoacid produced in the reaction. Furthermore, the halogen oxoacid may also be decomposed by light, and thus the reaction tube, accompanying pipes, and the like are preferably light-shielded.

In the present embodiment, when an organic solvent is used as the solvent, the reaction apparatus with an explosion-proof structure is preferably provided. Therefore, in order to simplify the apparatus configuration, water is preferably used as the solvent for the organic alkaline solution.

In the present embodiment, organic polymer materials used for the inner surface of the reaction tube include vinyl chloride resin (flexible or rigid vinyl chloride resin), nylon resin, silicone resin, polyolefin resin (polyethylene, polypropylene), and fluororesin. Among these organic polymer materials, the fluororesin is preferable from the viewpoint of ease of molding, resistance to solvents, low elution of impurities, and the like.

The fluororesin is not particularly limited as long as the fluororesin is a resin (polymer) containing fluorine atoms, and any known fluororesin can be used. Examples of the fluororesin include polytetrafluoroethylene, polychlorotrifluoroethylene, polyvinylidene fluoride, tetrafluoroethylene-hexafluoropropylene copolymer, tetrafluoroethylene-perfluoroalkyl vinyl ether copolymer, tetrafluoroethylene-ethylene copolymer, chlorotrifluoroethylene-ethylene copolymer, and cyclic perfluoro (butenyl vinyl ether) polymer. Among these, the tetrafluoroethylene-perfluoroalkyl vinyl ether copolymer is preferably used from the viewpoint of availability of the reaction tube, productivity, and the like.

In the present embodiment, examples of a method for forming the inner surface of the reaction tube by an organic polymer material include a method in which the entire reaction tube is formed by an organic polymer material, and a method in which only the inner surface of a reaction tube made of glass or stainless steel is covered with an organic polymer material.

The inner surface can be cleaned before use to prevent metal components from eluting from the organic polymer material. Specifically, it is preferable to sufficiently clean the inner surface with acid such as high-purity nitric acid and hydrochloric acid (for example, immerse the reaction tube in a solution having an acid concentration of 1 mol/L for 12 hours to clean the inner surface), and further clean the inner surface with ultrapure water and the like. In order to achieve a stable reaction, it is preferable to clean the inner surface of the reactor formed from the organic polymer material by the method described above, before causing the organic alkaline solution to react with the chlorine gas.

In the present embodiment, as long as the surface contacted by the organic alkaline solution in the reaction tube is formed of an organic polymer material, the other portions of the reaction tube can be formed of glass, stainless steel, or passivated stainless steel.

In the present embodiment, it is only required to bring the organic alkaline solution and the chlorine gas into contact with each other in the reaction tube, and the range of the reaction temperature during the reaction is not particularly limited, but is preferably similar to the reaction temperature mentioned above. When carbon dioxide is present in the reaction system, the pH of the obtained halogen oxoacid solution tends to decrease. Therefore, in consideration of stable manufacturing, it is preferable that the reaction system does not include carbon dioxide. Specifically, it is preferable to use an organic alkaline solution, chlorine gas, and the like in which the amount of carbon dioxide is reduced. The reaction is preferably implemented in the presence of an inert gas (for example, in the presence of nitrogen gas) in which the amount of carbon dioxide is reduced. When the reaction occurs under such conditions, it is possible to suppress a decrease of the pH of the obtained halogen oxoacid solution, and thus, the storage stability is improved.

### Halogen Oxoacid Manufacturing Apparatus

Next, an embodiment of a halogen oxoacid manufacturing apparatus will be described. The manufacturing method described above can be implemented by using the manufacturing apparatus according to the present embodiment. In an example of the manufacturing apparatus according to the present embodiment, a quaternary alkylammonium hydroxide solution is used as the organic alkali, and chlorine gas is used as the halogen. Note that conditions such as the type, the concentration, and the like of the organic alkali and the halogen supplied as raw materials can be the same as the conditions described in the manufacturing method of the halogen oxoacid described above.

The manufacturing apparatus according to the present embodiment includes a reaction tube having an axis extending from a first end to a second end of the reaction tube, the reaction tube extending in the axial direction while circling around the axis. The reaction tube with the axis extending substantially in a horizontal direction is configured to continuously supply an organic alkaline solution and a halogen from the first end to the second end of the reaction tube so that liquid phase parts and gas phase parts are alternately and repeatedly provided in a transfer passage of the reaction tube, to perform gas-liquid mixing of the organic alkaline solution and the halogen at the liquid phase parts and/or gas phase parts.

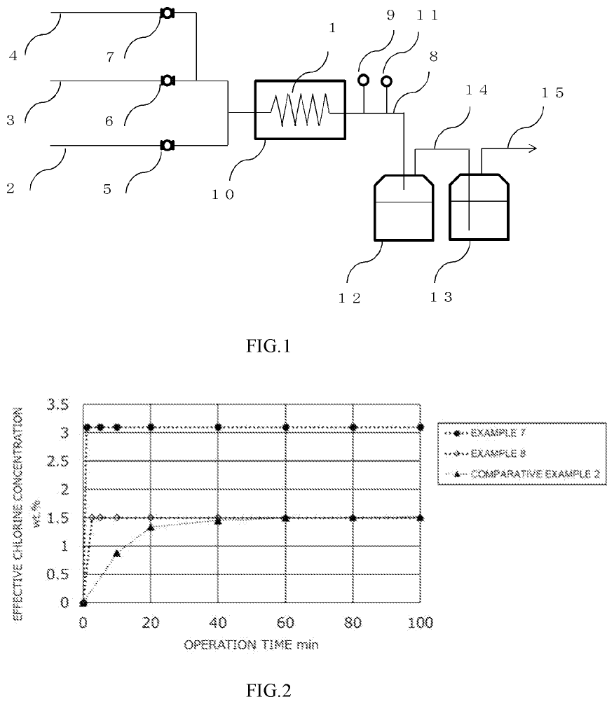

FIG. 1 is a schematic view of the manufacturing apparatus according to the present embodiment. The manufacturing apparatus described in FIG. 1 includes a reaction tube 1, a supply pipe 2 for a quaternary alkylammonium hydroxide solution as a supply means of an organic alkali to the reaction tube, a pipe valve 5 for performing a liquid supply operation and a stop operation, a chlorine gas supply pipe 3 as a supply means of a halogen, and a pipe valve 6 for performing a supply operation and a stop operation of chlorine gas, and further includes a reaction solution removal pipe 8 as a reaction solution removal means for removing a reaction solution from the reaction tube to the outside.

In the reaction tube 1, the quaternary alkylammonium hydroxide solution to be supplied is supplied through the supply pipe 2 for the quaternary alkylammonium hydroxide solution, and the chlorine gas to be supplied is supplied through the chlorine gas supply pipe 3, and both the quaternary alkylammonium hydroxide solution and the chlorine gas are supplied continuously. The supply operation and the stop operation of the chlorine gas are performed by using the pipe valve 6. The generated reaction solution is continuously removed through the reaction solution removal pipe 8.

The same conditions as described above in the manufacturing method can be used for a location where the quaternary alkylammonium hydroxide solution flows and contacts the reaction tube 1. An inner surface of the reaction tube 1 is preferably formed of an organic polymer material. Specifically, the reaction tube preferably includes a fluororesin, and the entire reaction tube 1 or at least the inner surface of the reaction tube 1 is preferably made of a fluororesin. The fluororesin can be any of those indicated in “Material of Inner Surface of Reaction Tube”.

Preferably, the chlorine gas supply pipe 3 used as a chlorine gas supply means and the reaction tube 1 have the same tube diameter, and in order to facilitate dispersion of the chlorine gas into the liquid phase, the tube diameter of the chlorine gas supply pipe 3 can be smaller than the tube diameter of the reaction tube 1. A blowing speed of the chlorine gas is preferably 0.1 m/sec or more and 10 m/sec or less in terms of the chlorine gas at 0° C. and 1 atm.

The volume in the reaction tube can vary depending on the supply speed of the quaternary alkylammonium hydroxide solution used. When a value obtained by dividing the volume of the reaction tube by the volume of the quaternary alkylammonium hydroxide solution supplied to the reaction tube per time unit is defined as the liquid retention time, the reaction tube preferably has such a volume that the liquid retention time of the quaternary alkylammonium hydroxide solution is preferably from 5 seconds to 30 minutes, and more preferably from 10 seconds to 5 minutes. The supply speed of the halogen, expressed as a ratio of the volume flow rate of the halogen to the volume flow rate of the organic alkaline solution supplied to the reaction tube, is preferably from 1 to 50, and more preferably from 10 to 30.

When a direction extending from the first end to the second end of the reaction tube is defined as an axis, the reaction tube preferably extends in the axial direction while circling around the axis. The reaction tube is preferably arranged to extend substantially in the horizontal direction, and more preferably, the reaction tube is formed in a spiral shape, and is arranged so that a spiral axis of the reaction tube extends substantially in the horizontal direction. The diameter of the revolution required for circling (inner diameter of the spirally formed circles) can be determined depending on the length of the reaction tube and the strength of the material used for the reaction tube, and examples thereof include diameters of 30 mm or more and 3000 mm or less, and preferably 60 mm or more and 600 mm or less. When the reaction tube circles around one or more times, an effect of improving the gas-liquid mixing is obtained. Therefore, the number of revolutions of the reaction tube has no upper limit, but preferably, an apparatus is formed to have at least 2 or more revolutions, more preferably 5 or more revolutions, and even more preferably 10 or more revolutions. On the other hand, the upper limit of the number of revolutions is usually 50 revolutions or less. A higher number of revolutions of the reaction tube is advantageous for the mixing of the gas and the liquid. The range of the average inner diameter of the reaction tube has no upper limit, but is preferably 5 mm or more, more preferably 5 mm or more and 500 mm or less, and even more preferably 10 mm or more and 100 mm or less.

In the reaction tube 1, in order to adjust the concentration of the gas component supplied to the reaction tube 1, a nitrogen gas supply pipe 4 can be provided as a means for supplying nitrogen into the reaction tube 1. Additionally, a pipe valve 7 can be provided to perform a supply operation and a stop operation of the nitrogen gas. The reaction between the quaternary alkylammonium hydroxide solution and chlorine is an exothermic reaction. In the manufacturing apparatus according to the present embodiment, the temperature in the reaction tube 1 can be measured by using, for example, a reaction solution temperature measurement device 9 as a temperature measurement means in the reaction tube 1. The manufacturing apparatus according to the present embodiment can include a reaction tube temperature control jacket 10 as a reaction temperature control means in the reaction tube, specifically, a means for removing heat from the reaction tube. The reaction tube temperature control jacket 10 can remove heat. The manufacturing apparatus according to the present embodiment can include a light-shielding means to realize the reaction under light-shielding conditions.

The manufacturing apparatus according to the present embodiment can further include a reaction solution pH measurement device 11 arranged in the reaction solution removal pipe 8, as a pH measurement means for the reaction solution. A branched line is more preferably used to pass a measurement solution used in the pH measurement, because the measurement solution may cause contamination of the reaction solution. Using the reaction solution pH measurement device 11 makes it possible to measure the pH of the reaction solution after the reaction. The manufacturing apparatus according to the present embodiment is preferably provided with at least one, more preferably with two, and even more preferably with all of the reaction temperature measurement means, the reaction temperature control means, and the pH measurement means in the reactor described above.

A detoxification means can be provided downstream of the reaction solution removal pipe 8 to prevent unreacted chlorine or the like from being discharged out of the system. Specifically, a caustic soda detoxification device 13 can be provided as the detoxification means.

For example, the caustic soda detoxification device 13 can have a configuration in which gas included in the reaction solution, which is generated in a gas phase part of a reaction solution tank 12 for storing a reaction solution transported through the reaction solution removal pipe 8, is transported to the caustic soda detoxification device 13, via an exhaust gas pipe 14 connected to the tank, and injected into a solution including caustic soda. Additionally, the gas phase part of the caustic soda detoxification device 13 can include an exhaust gas pipe 15 that discharges harmless gas.

### EXAMPLES

Next, the present invention will be described in detail using examples and comparative examples, but the present invention is not limited to these examples.

### (pH Measurement Method)

The pH of 30 mL of a quaternary alkylammonium hydroxide solution and a quaternary alkylammonium hypochlorite solution was measured using a tabletop pH meter (LAQUA F-73, manufactured by HORIBA, Ltd.). The pH measurement was performed after stabilizing the solution at 25° C.

Calculation Method of Effective Chlorine Concentration and Hypochlorite Ion Concentration 0.5 mL of the treated solution (quaternary alkylammonium hypochlorite solution), 2 g of potassium iodide (manufactured by Fujifilm Wako Pure Chemical Corporation, special grade reagent), 8 mL of 10 mass % acetic acid, and 10 mL of ultrapure water were placed into a 100 mL Erlenmeyer flask, and stirred until solids were dissolved to obtain a brown solution.

The prepared brown solution was subjected to redox titration by using a 0.02 M sodium thiosulfate solution (manufactured by Fujifilm Wako Pure Chemical Corporation, volumetric analysis grade), until the color of the solution changed from brown to very light yellow, and then, a starch solution was added to obtain a solution of light purple color.

The 0.02 M sodium thiosulfate solution was further added to this solution, and the effective chlorine concentration was calculated using a point where the solution turned colorless and transparent as the end point. The hypochlorite ion concentration was calculated from the obtained effective chlorine concentration. For example, when the effective chlorine concentration is 1 mass %, the hypochlorite ion concentration is 0.73 mass %.

### (Chlorine Yield)

The chlorine yield was determined from the ratio (%) of the number of moles of hypochlorite ions produced relative to the number of moles of chlorine molecules supplied into the organic alkaline solution. When the whole amount of chlorine added to the organic alkaline solution undergoes reaction (and no decomposition occurs), the chlorine yield is 100%. The chlorine yield decreases when hypochlorite ions decompose during the reaction.

### (Evaluation Method of Storage Stability)

The quaternary alkylammonium hypochlorite solution was transferred into a glove bag, and after the carbon dioxide concentration in the glove bag reached 1 ppm or less, the solution was transferred to a container made of perfluoroalkoxy (PFA) fluororesin (tetrafluoroethylene-perfluoroalkyl vinyl ether copolymer), and the container was sealed. Subsequently, after 10 days of storage in a light-shielded environment at 23° C., the hypochlorite ion concentration of the quaternary alkylammonium hypochlorite solution in the PFA container was measured. A hypochlorite ion concentration ratio (concentration after 10 days/initial concentration) from 80% to 100% was defined as good, a concentration ratio of 60% or greater and less than 80% was defined as satisfactory, and a concentration ratio below 60% was defined as poor.

### Example 1

A reaction tube made of PFA which is a fluororesin (inner diameter 8 mm, length 1 m) was used to form a spiral shaped reaction tube which circles six times around an axis extending in the horizontal direction. The diameter of the spiral shape was 50 mm and the reaction tube was arranged in the horizontal direction. A tetramethylammonium hydroxide solution (concentration of 12.0 mass %, pH 14.1, liquid temperature 5° C.) and chlorine were supplied from an inlet side of the reaction tube at rates of 370 mL/min and 209.8 mmol/min, respectively. For the first one minute from the start of the operation, the reaction solution obtained from an outlet side of the reaction tube was discarded in order to replace the reaction solution in the reaction tube. Thereafter, the reaction solution was sampled continuously for 5 minutes. The liquid retention time was 8 seconds, and the gas phase parts and the liquid phase parts were alternately present in the transfer direction in the reaction tube. The amount of unreacted chlorine was calculated based on the effective chlorine concentration in the sodium hydroxide solution (concentration of 10 mass %, 1000 mL) used for preventing chlorine leakage as a downstream process of the reaction tube, and was 100 mass ppm or less of the total supplied chlorine amount. As a result, a quaternary tetramethylammonium hypochlorite solution (effective chlorine concentration of 3.9 mass %, pH 13.4, liquid temperature 17° C.) was obtained. The chlorine yield was 99% or greater and the storage stability was good.

Furthermore, after the sampling described above, the operation was continued during 2 hours under the same conditions. In the liquid composition of a sample obtained by performing a 5 minute sampling one hour after the operation start, an effective chlorine concentration was 3.9 mass % and the pH value was 13.4, and in the liquid composition of a sample obtained by performing a 5 minute sampling two hours after the operation start, an effective chlorine concentration is 3.9 mass % and the pH value was 13.4. The production amount per hour was 23 L/H.

### Comparative Example 1

In a comparative example, an experimental example is described in which the reaction tube used in Example 1 is not processed.

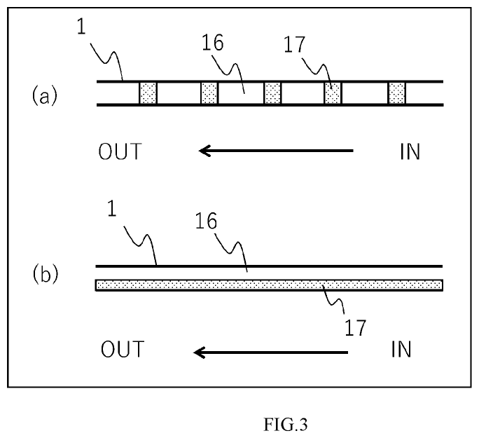

A reaction tube (inner diameter 8 mm, length 1 m) made of PFA, which is a fluororesin, was extended into a straight tube shape and arranged in a horizontal direction. The tetramethylammonium hydroxide solution (concentration of 12.0 mass %, pH 14.1, liquid temperature 5° C.) and chlorine were supplied from one end of the reaction tube at rates of 370 mL/min and 209.8 mmol/min, respectively. The liquid supply became unstable due to generation of a short path of the chlorine gas in the reaction tube, and decreased to 140 mL/min. The liquid retention time was 22 seconds, and in the reaction tube, the gas phase part and the liquid phase part were separated into an upper layer and a lower layer, and were not present alternately in the transfer direction (see FIG. 3(b)). The reaction solution obtained from the outlet side of the reaction tube was sampled continuously for 2 minutes. The amount of unreacted chlorine was calculated based on the effective chlorine concentration in the sodium hydroxide solution (concentration of 10 mass %, 1000 mL) used for preventing chlorine leakage as a downstream process, and was 50% of the total supplied chlorine amount. As a result, a quaternary tetramethylammonium hypochlorite solution (effective chlorine concentration of 4.5 mass %, pH 11.0, liquid temperature 18° C.) was obtained. The chlorine yield was 90% and the storage stability was poor.

### Example 2

A reaction tube made of PFA which is a fluororesin (inner diameter 8 mm, length 1 m) was used to form a spiral shaped reaction tube which circles three and a half times around an axis extending in the horizontal direction. The diameter of the spiral shape was 100 mm and the reaction tube was arranged in the horizontal direction. A tetramethylammonium hydroxide solution (concentration of 4.8 mass %, pH 13.7, liquid temperature 5° C.) and chlorine were supplied from an inlet side of the reaction tube at rates of 200 mL/min and 12.5 mmol/min, respectively. For the first one minute from the start of the operation, the reaction solution obtained from an outlet side of the reaction tube was discarded in order to replace the reaction solution in the reaction tube. Thereafter, the reaction solution was sampled continuously for 5 minutes. The liquid retention time was 15 seconds, and the gas phase parts and the liquid phase parts were alternately present in the transfer direction in the reaction tube. The amount of unreacted chlorine was calculated based on the effective chlorine concentration in the sodium hydroxide solution (concentration of 10 mass %, 1000 mL) used for preventing chlorine leakage as a downstream process of the reaction tube, and was 500 mass ppm of the total supplied chlorine amount. As a result, a quaternary tetramethylammonium hypochlorite solution (effective chlorine concentration of 0.4 mass %, pH 13.6, liquid temperature 6° C.) was obtained. The chlorine yield was 99% or greater and the storage stability was good.

### Example 3

An experimental example is described in which the reaction tube used in Example 2 is not processed.

A reaction tube (inner diameter 8 mm, length 1 m) made of PFA, which is a fluororesin, was extended into a straight tube shape and arranged in a horizontal direction. A tetramethylammonium hydroxide solution (concentration of 4.8 mass %, pH 13.7, liquid temperature 7° C.) and chlorine were supplied from one end of the reaction tube at rates of 200 mL/min and 12.5 mmol/min, respectively. For the first one minute from the start of the operation, the reaction solution obtained from an outlet side of the reaction tube was discarded in order to replace the reaction solution in the reaction tube. Thereafter, the reaction solution was sampled continuously for 5 minutes. The liquid retention time was 15 seconds, and the gas phase parts and the liquid phase parts were alternately present in the transfer direction in the reaction tube (see FIG. 3(a)). The amount of unreacted chlorine was calculated based on the effective chlorine concentration in the sodium hydroxide solution (concentration of 10 mass %, 1000 mL) used for preventing chlorine leakage as a downstream process of the reaction tube, and was 5% of the total supplied chlorine amount. As a result, a quaternary tetramethylammonium hypochlorite solution (effective chlorine concentration of 0.4 mass %, pH 13.6, liquid temperature 8° C.) was obtained. The chlorine yield was 99% or greater and the storage stability was good.

### Example 4

The same spiral-shaped reaction tube as used in Example 1 was arranged in the horizontal direction. A tetramethylammonium hydroxide solution (concentration of 25.0 mass %, pH 14.4, liquid temperature 6° C.) and chlorine were supplied from an inlet side of the reaction tube at rates of 75 mL/min and 75.9 mmol/min, respectively. For the first one minute from the start of the operation, the reaction solution obtained from an outlet side of the reaction tube was discarded in order to replace the reaction solution in the reaction tube. Thereafter, the reaction solution was sampled continuously for 5 minutes. The liquid retention time was 40 seconds, and the gas phase parts and the liquid phase parts were alternately present in the transfer direction in the reaction tube. The amount of unreacted chlorine was calculated based on the effective chlorine concentration in the sodium hydroxide solution (concentration of 10 mass %, 1000 mL) used for preventing chlorine leakage as a downstream process of the reaction tube, and was 0.2% of the total supplied chlorine amount. As a result, a quaternary tetramethylammonium hypochlorite solution (effective chlorine concentration of 6.7 mass %, pH 13.8, liquid temperature 30° C.) was obtained. The chlorine yield was 99% or greater and the storage stability was good.

### Example 5

An experimental example is described in which the reaction tube used in Example 4 is not processed.

The same straight tube-shaped reaction tube as used in Comparative Example 1 was arranged in the horizontal direction. A tetramethylammonium hydroxide solution (concentration of 25.0 mass %, pH 14.4, liquid temperature 6° C.) and chlorine were supplied from one end of the reaction tube at rates of 75 mL/min and 75.9 mmol/min, respectively. For the first one minute from the start of the operation, the reaction solution obtained from an outlet side of the reaction tube was discarded in order to replace the reaction solution in the reaction tube. Thereafter, the reaction solution was sampled continuously for 5 minutes. The liquid retention time was 40 seconds, and the gas phase parts and the liquid phase parts were alternately present in the transfer direction in the reaction tube. The amount of unreacted chlorine was calculated based on the effective chlorine concentration in the sodium hydroxide solution (concentration of 10 mass %, 1000 mL) used for preventing chlorine leakage as a downstream process of the reaction tube, and was 13% of the total supplied chlorine amount. As a result, a quaternary tetramethylammonium hypochlorite solution (effective chlorine concentration of 6.0 mass %, pH 14.0, liquid temperature 27° C.) was obtained. The chlorine yield was 99% or greater and the storage stability was satisfactory.

### Example 6

A reaction tube made of PTFE which is a fluororesin (inner diameter 11 mm, length 10 m) was used to form a spiral shaped reaction tube which circles 20 times around an axis extending in the horizontal direction. The diameter of the spiral shape was 150 mm. The reaction tube was arranged in the horizontal direction in a container filled with cold water to also perform a cooling operation. Subsequently, a tetramethylammonium hydroxide solution (concentration of 25 mass %, pH 14.4, liquid temperature 5° C.) and chlorine were supplied from an inlet side of the reaction tube at rates of 50 mL/min and 68.3 mmol/min, respectively. For the first 20 minutes from the start of the operation, the reaction solution obtained from an outlet side of the reaction tube was discarded in order to replace the reaction solution in the reaction tube. Thereafter, the reaction solution was sampled continuously for 5 minutes. The liquid retention time was 19 minutes, and the gas phase parts and the liquid phase parts were alternately present in the transfer direction in the reaction tube. The amount of unreacted chlorine was calculated based on the effective chlorine concentration in the sodium hydroxide solution (concentration of 10 mass %, 1000 mL) used for preventing chlorine leakage as a downstream process of the reaction tube, and was 10 ppm or less of the total supplied chlorine amount. As a result, a quaternary tetramethylammonium hypochlorite solution (effective chlorine concentration of 8.8 mass %, pH 12.0, liquid temperature 15° C.) was obtained. The chlorine yield was 99% or greater and the storage stability was good.

### Example 7

The same spiral-shaped reaction tube as used in Example 6 was arranged in the horizontal direction. A tetramethylammonium hydroxide solution (concentration of 10.0 mass %, pH 14.0, liquid temperature 15° C.) and chlorine were supplied from the inlet side of the reaction tube at rates of 2.5 L/min and 1.12 mol/min, respectively, and a reaction solution obtained from the outlet side of the reaction tube was continuously sampled after starting the supply of chlorine. The liquid retention time was 20 seconds, and a quaternary tetramethylammonium hypochlorite solution (effective chlorine concentration of 3.1 mass %, pH 13.3, liquid temperature 25° C.) was stably obtained after the elapse of one minute from the start of the supply of chlorine, as shown in FIG. 2. The gas phase parts and the liquid phase parts were alternately present in the transfer direction in the reaction tube. The amount of unreacted chlorine was calculated based on the effective chlorine concentration in the sodium hydroxide solution (concentration of 10 mass %, 1000 mL) used for preventing chlorine leakage as a downstream process of the reaction tube, and was 100 mass ppm or less of the total supplied chlorine amount. The chlorine yield was 99% or greater and the storage stability was good.

### Example 8

A reaction tube made of PTFE which is a fluororesin (inner diameter 11 mm, length 3 m) was used to form a spiral shaped reaction tube which circles six times around an axis extending in the horizontal direction. The diameter of the spiral shape was 150 mm and the reaction tube was arranged in the horizontal direction. A tetramethylammonium hydroxide solution (concentration of 5.0 mass %, pH 13.7, liquid temperature 12° C.) and chlorine were supplied from the inlet side of the reaction tube at rates of 110 mL/min and 24.3 mmol/min, respectively, and a reaction solution obtained from the outlet side of the reaction tube was continuously sampled after starting the supply of chlorine. The liquid retention time was 2 minutes, and a quaternary tetramethylammonium hypochlorite solution (effective chlorine concentration of 1.5 mass %, pH 13.0, liquid temperature 17° C.) was stably obtained after the elapse of 3 minutes from the start of the supply of chlorine, as shown in FIG. 2. The gas phase parts and the liquid phase parts were alternately present in the transfer direction in the reaction tube. The volume of a waste liquid generated in the replacement operation until the reaction was stable was 0.2 L, and the amount of the unreacted chlorine was 100 mass ppm or less of the total supplied chlorine amount.

### Comparative Example 2

A cylindrical reaction tube (made of PTFE) having a diameter of 150 mm was used and 1700 mL of a tetramethylammonium hydroxide solution (5.0 mass %, pH 13.7, liquid temperature 12° C.) were placed into the reaction tube, and the tetramethylammonium hydroxide solution having the same composition and chlorine gas were supplied to the reactor at rates of 110 mL/min and 24.3 mmol/min, respectively. The generated tetramethylammonium hypochlorite solution was extracted through a liquid extraction port provided at a height of 100 mm from a bottom of the reactor. The liquid retention time was 16 minutes, and a quaternary tetramethylammonium hypochlorite solution (effective chlorine concentration of 1.5 mass %, pH 13.0, liquid temperature 17° C.) was stably obtained after the elapse of 60 minutes from the start of the supply of chlorine, as shown in FIG. 2. In the reaction tube, the gas phase part and the liquid phase part were separated into an upper layer and a lower layer, and were not present alternately in the transfer direction. The volume of the waste liquid generated in the replacement operation until the reaction was stable was 6.7 L, and the amount of the unreacted chlorine was 2% of the total supplied chlorine amount.

### Example 9

The same spiral-shaped reaction tube as used in Example 8 was arranged in the horizontal direction, and a tetramethylammonium hydroxide solution (concentration of 8.5 mass %, pH 14.0, liquid temperature 10° C.) and chlorine were supplied from the inlet side of the reaction tube, at rates of 1000 mL/min and 436 mmol/min, respectively. The gas phase parts and the liquid phase parts were alternately present in the transfer direction in the reaction tube. The amount of the quaternary tetramethylammonium hypochlorite solution (effective chlorine concentration of 3.0 mass %, pH 13.0, liquid temperature 20° C.) obtained from the outlet side of the reaction tube, per hour, was 62 L/H.

### Comparative Example 3

A quaternary tetramethylammonium hypochlorite solution (effective chlorine concentration of 3.0 mass %, pH 13.0, liquid temperature 5° C.) was obtained by using the manufacturing method of Example 3 of the related art (WO 2019/225541). In this manufacturing method, the reaction time was 180 minutes, and the production amount per hour was 0.34 L/H.

TABLE 1

| | Inner | | | Number of | | | | |
|---|---|---|---|---|---|---|---|---|
| | diameter | | | revolutions of | | | | |
| | of reaction | Length of | Outer appearance | reaction tube | Tetramethylammonium | | | |
| | tube | reaction tube | of reaction tube | Number of | hydroxide solution | | | Chlorine |
| | mm | m | — | circles | wt. % | pH | mL/min | mmol/min |
| Example 1 | 8 | 1 | Spiral shape | 6 | 12.0 | 14.1 | 370 | 209.8 |
| Comparative | 8 | 1 | Straight tube | 0 | 12.0 | 14.1 | 140 | 209.8 |
| Example 1 | | | | | | | | |
| Example 2 | 8 | 1 | Spiral shape | 3.5 | 4.8 | 13.7 | 200 | 12.5 |
| Example 3 | 8 | 1 | Straight tube | 0 | 4.8 | 13.7 | 200 | 12.5 |
| Example 4 | 8 | 1 | Spiral shape | 6 | 25 | 14.4 | 75 | 75.9 |
| Example 5 | 8 | 1 | Straight tube | 0 | 25 | 14.4 | 75 | 75.9 |
| Example 6 | 11 | 10 | Spiral shape | 20 | 25 | 14.4 | 50 | 68.3 |
| Example 7 | 11 | 10 | Spiral shape | 20 | 10 | 14.0 | 2500 | 1120 |

| | | | | |
|---|---|---|---|---|
| Liquid | | | | |
| retention | Liquid-gas | Chlorine | Unreacted | Storage |
| time | ratio | yield | chlorine | stability |
| — | — | % | % | — |

| | | | | | | |
|---|---|---|---|---|---|---|
| Example 1 | 8 | sec | 13 | ≥99 | 0.1≥ | Good |
| Comparative | 22 | sec | 34 | 92 | 50 | Poor |
| Example 1 | | | | | | |
| Example 2 | 15 | sec | 1 | ≥99 | 0.1≥ | Good |
| Example 3 | 15 | sec | 1 | ≥99 | 5 | Good |
| Example 4 | 40 | sec | 23 | ≥99 | 0.2 | Good |
| Example 5 | 40 | sec | 23 | ≥99 | 13 | Satisfactory |
| Example 6 | 19 | min | 31 | ≥99 | 0.1≥ | Good |
| Example 7 | 20 | sec | 10 | ≥99 | 0.1≥ | Good |

*The “liquid-gas ratio” in the table is the ratio of the volume flow rate of the halogen (chlorine gas) to the volume flow rate of the organic alkaline solution (tetramethylammonium hydroxide solution).

### REFERENCE SIGNS LIST

1 Reaction tube
2 Quaternary alkylammonium hydroxide solution supply pipe
3 Chlorine gas supply pipe
4 Nitrogen gas supply pipe
5 Pipe valve
6 Pipe valve
7 Pipe valve
8 Reaction solution removal pipe
9 Reaction solution temperature measurement device
10 Reaction tube temperature control jacket
11 Reaction solution pH measurement device
12 Reaction solution tank
13 Caustic soda detoxification device
14 Exhaust gas pipe
15 Exhaust gas pipe
16 Gas phase part
17 Liquid phase part

1. A method of manufacturing a halogen oxoacid, the method comprising:
continuously supplying an organic alkaline solution and a halogen from a first end to a second end of a reaction tube so that liquid phase parts and gas phase parts are alternately and repeatedly provided in a transfer passage of the reaction tube, to perform gas-liquid mixing of the organic alkaline solution and the halogen at the liquid phase parts and/or gas phase parts.

2. The method of manufacturing a halogen oxoacid according to claim 1, wherein a ratio of a volume flow rate of the halogen to a volume flow rate of the organic alkaline solution supplied to the reaction tube is from 1 to 50.

3. The method of manufacturing a halogen oxoacid according to claim 1, wherein the reaction tube has an axis extending from the first end to the second end of the reaction tube and extends in an axial direction while circling around the axis.

4. The method of manufacturing a halogen oxoacid according to claim 1, wherein the reaction tube is arranged to extend substantially in a horizontal direction.

5. The method of manufacturing a halogen oxoacid according to claim 1, wherein the reaction tube has an axis extending from the first end to the second end of the reaction tube, and the reaction tube is formed in a spiral shape having a spiral axis coinciding with the axis.

6. The method of manufacturing a halogen oxoacid according to claim 1, wherein the reaction tube contains a fluororesin.

7. The method of manufacturing a halogen oxoacid according to claim 1, wherein an average inner diameter of the reaction tube is 5 mm or greater.

8. A halogen oxoacid manufacturing apparatus, comprising:
a reaction tube having an axis extending from a first end to a second end of the reaction tube, the reaction tube extending in an axial direction while circling around the axis, wherein
the reaction tube with the axis extending substantially in a horizontal direction is configured to continuously supply an organic alkaline solution and a halogen from the first end to the second end of the reaction tube so that liquid phase parts and gas phase parts are alternately and repeatedly provided in a transfer passage of the reaction tube, to perform gas-liquid mixing of the organic alkaline solution and the halogen at the liquid phase parts and/or gas phase parts.

9. The halogen oxoacid manufacturing apparatus according to claim 8, further comprising a supplying unit configured to supply, to the reaction tube, the organic alkaline solution and the halogen with a ratio of a volume flow rate of the halogen to a volume flow rate of the organic alkaline solution from 1 to 50.

10. The halogen oxoacid manufacturing apparatus according to claim 8, wherein the reaction tube is a reaction tube formed in a spiral shape, and a spiral axis of the reaction tube extends substantially in the horizontal direction.

11. The halogen oxoacid manufacturing apparatus according to claim 8, wherein the reaction tube contains a fluororesin.

12. The halogen oxoacid manufacturing apparatus according to claim 8, wherein an average inner diameter of the reaction tube is 5 mm or more.

13. The halogen oxoacid manufacturing apparatus according to claim 8, wherein the reaction tube is configured to achieve a liquid retention time of the organic alkaline solution of 5 seconds to 30 minutes.
